(19) United States
(12) Patent Application Publication
(10) Pub. No.: US 2012/0035702 A1
(43) Pub. Date: Feb. 9, 2012

(54) STENT FOR VALVE REPLACEMENT

(76) Inventors: Keith Horvath, Washington, DC (US); Dumitru Mazilu, Lutherville, MD (US); Ming Li, Potomac, MD (US)

(21) Appl. No.: 13/265,315

(22) PCT Filed: Apr. 23, 2010
(86) PCT No.: PCT/US10/32257

§ 371 Date: Oct. 19, 2011

Related U.S. Application Data
(60) Provisional application No. 61/172,568, filed on Apr. 24, 2009.

(51) Int. Cl. A61F 2/84; A61F 2/82

(52) U.S. Cl. 623/1.11; 623/1.16; 623/1.34; 623/1.46

(57) ABSTRACT

An expandable stent (12) for use in the implantation of a valve prosthesis (11) using a delivery system (10) is disclosed. The self-expandable stent (12) includes a tubular lattice structure (13) defined by longitudinally aligned rods (16) connected to V- shaped struts (17) for forming a plurality of interconnected chevron-shaped six-sided polygons (24) that define a distal end zone (21) and a middle zone (20) of the tubular lattice structure (13). A flare (27) or bend may be defined along opposite ends of each rod (16) to properly seat and prevent undue torsion of the stent (12) and valve prosthesis (11) during deployment and placement of the stent (12) within the lumen.

### STATEMENT OF FEDERALLY SPONSORED RESEARCH

The U.S. Government has certain rights in the invention described herein, which was made in part with funds from NM Contract No. HHSN263200700191.

### FIELD

The present document relates to stents used to deliver and position valve prostheses within the human anatomy.

### BACKGROUND

Aortic stenosis and aortic regurgitation are the most common types of aortic valvular diseases. When treating aortic valvular diseases, a diseased natural valve in the body is traditionally replaced with a valve prosthesis by surgical implantation.

Two basic types of artificial aortic valves are available for replacement of diseased human heart valves. The first type, a mechanical valve, is constructed of synthetic rigid materials, such as polymer or metal. Its use is associated with thrombogenesis, which requires valve recipients to be on long term anti-coagulants. The second type, a tissue valve or bioprosthetic valve, includes valve leaflets of preserved animal tissue mounted on an artificial support or “stent.”

Presently, an aortic valve replacement procedure requires a sternotomy and the use of cardiopulmonary bypass to arrest the heart and provide a bloodless field in which to operate. The native aortic valve is resected through a large incision in the aorta and then a prosthetic valve is sutured in the place of the native valve. Due to the invasiveness of the procedure, aortic valve replacement surgery is associated with significant risk of morbidity and mortality, especially in elderly patients.

To decrease the risks associated with aortic valve replacement procedures, many surgeons and scientists have pursued less invasive approaches and techniques. There are two methods that are currently being investigated and developed for minimally invasive aortic valve replacement: percutaneous transcatheter valve delivery and transapical aortic valve replacement. The latter approach is emerging as a viable minimally invasive approach that consists of the placement of a bioprosthetic valve via a trocar that is inserted into the apex of the beating heart. Generally, the prosthesis used for both types of techniques includes a prosthetic valve affixed or sewn into a balloon-expandable or self-expanding stent that is surgically implanted.

However, the durability of bioprosthetic heart valves is limited to about 12 to 15 years. The limitations in the long term performance of bioprosthetic heart valves are believed to be due largely to the mechanical properties of the valve and the stresses imposed on the tissue leaflets by the rigidity of the stent structure while the aortic root to which the artificial valve is attached expands and contracts during the cardiac cycle. An important feature of the natural heart valve is its ability to expand in diameter by more than 10% during systole. This ability of the aortic root to expand facilitates blood flow due to a better opening of the valve during systole and contributes to minimal bending of the cusps, thus reducing possible internal flexural fatigue. In addition to the issue of expansion/contraction of the aortic root, there is also significant torsion/twisting motion that the aorta undergoes during each pulse. Ideally, this motion needs to be accounted for by any prosthetic valve design that is anchored or affixed to the aortic wall.

Other artificial valve designs have attempted to overcome the rigidity of artificial heart valves and accommodate the expansion of the aortic root during systole. Although these types of artificial valve designs allow for improved hemodynamics, such designs have not totally solved the problems arising from the rigidity of artificial heart valve stents.

Therefore, there is a need for a stent that is expandable, resilient, and durable, and that can be delivered and repositioned in a patient in need thereof, particularly a patient in need of an aortic valve replacement, while providing a better opening of the valve during systole to facilitate blood flow and contributing to minimal bending of the cusps to reduce valve failure.

### SUMMARY

In one embodiment, a stent includes a tubular lattice structure having a radial direction and a longitudinal direction. The tubular lattice structure defines a middle zone in communication with a proximal end zone and a distal end zone. The proximal end zone includes a plurality of interconnected four-sided polygons and the middle zone and distal end zone includes a plurality of rods positioned substantially in the longitudinal direction of the tubular lattice structure and interconnected by a plurality of struts that collectively define a plurality of six-sided polygons with each strut defining an apex that is oriented towards the proximal end zone.

In another embodiment, a delivery system includes a hollow outer sheath defining an opening with a self-expandable stent disposed adjacent the opening and in a collapsed form. The self-expandable stent further includes a tubular lattice structure having a radial direction and a longitudinal direction, with the tubular lattice structure adapted to assume a fully expanded form from a collapsed form after deployment of the self-expandable stent from the outer sheath. The tubular lattice structure defines a middle zone in communication with a proximal end zone and a distal end zone. The proximal end zone includes a plurality of interconnected four-sided polygons and the middle zone and distal end zone include a plurality of rods positioned substantially in the longitudinal direction of the tubular lattice structure and interconnected by a plurality of struts that collectively define a plurality of six-sided polygons with each strut defining an apex that is oriented towards the proximal end zone. A valve prosthesis is attached to the inside of the tubular lattice structure of the self-expandable stent.

In yet another embodiment, a method for delivering and repositioning a stent in a lumen includes providing a stent in a delivery system with the delivery system having a hollow, retractable hollow outer sheath defining an opening with the stent disposed therein adjacent the opening; constraining the stent in a collapsed form; delivering the stent percutaneously to a location in a lumen that requires repair or replacement; retracting the outer sheath relative to the stent and permitting the stent to expand from the collapsed form to a fully expandable form in the location; and monitoring the orientation and the location of the stent in the lumen.

Additional objectives, advantages and novel features will be set forth in the description which follows or will become apparent to those skilled in the art upon examination of the drawings and detailed description which follows.

### BRIEF DESCRIPTION OF THE DRAWINGS

FIG. 1 is an elevated perspective view of one embodiment of a stent;

FIG. 2 is a side elevation view of the stent shown in a fully expanded form;

FIG. 3 is partial side elevation view of the stent shown in the fully expanded form illustrating the flare defined at the proximal and distal ends of the stent;

FIG. 4 is a perspective view of the stent shown in a collapsed form;

FIG. 5 is an elevated perspective view of the stent in the fully expanded form shown attached to a valve prosthesis;

FIG. 6 is a perspective view of the stent in the fully expanded form shown attached to the valve prosthesis with a passive marker attached to one end of the stent;

FIG. 7 is a perspective view of the stent shown in the collapsed form and attached to the valve prosthesis disposed inside a delivery system;

FIG. 8 is a side view of the stent and valve prosthesis after deployment from the delivery system;

FIG. 9 is a perspective view of the stent attached to the valve prosthesis disposed inside the delivery system illustrating an active guide wire and a passive marker affixed to the stent;

FIG. 10 illustrates an image artifact shown in a cross-sectional view of a magnetic resonance imaging (MRI) image;

FIG. 11 illustrates another image artifact shown in a longitudinal sectional view of an MRI image;

FIG. 12 illustrates a colored trace on a cross-sectional view of an MRI image illustrating the active guide wire shown in FIG. 11 in red highlight;

FIG. 13 illustrates the colored trace on another MRI image of the active guide wire shown in FIG. 11;

FIG. 14 is a perspective view of the stent in collapsed form attached to the valve prosthesis disposed inside a manual delivery system;

FIG. 15 is a perspective view of the stent in collapsed form attached to the valve prosthesis disposed inside a robotic delivery system;

FIG. 16 is a side view showing the unfolded geometric configuration of the tubular lattice structure and a related isolated view of an embodiment of the rod with flared ends;

FIG. 17 is a perspective view of the stent shown in the fully expanded form;

FIG. 18 is a cross-sectional view of a middle zone of the stent shown in the fully expanded form;

FIG. 19 is a perspective view of the stent and the passive marker;

FIG. 19A is an enlarged view of the stent showing the passive marker affixed thereto;

FIG. 20 is a side view showing the unfolded geometric configuration of the stent illustrating a plurality of grasping members affixed to the stent;

FIG. 21 is a perspective view of the stent shown in the fully expanded form illustrating the flared first end zone and flared second end zone;

FIGS. 22A and 22B are partial perspective views of the stent showing the sequence of deployment of the stent from the delivery system;

FIG. 23 are radiographs of the anterior and right lateral aspects of the heart showing a well expanded stent in the aortic root;

FIG. 24 are images of valve sections that show widely patent coronary ostia being unobstructed by the stent or the commissures of the valve prosthesis after deployment; and

FIG. 25 is a side view of another embodiment of the stent.

Corresponding reference characters indicate corresponding elements among the view of the drawings. The headings used in the figures should not be interpreted to limit the scope of the claims.

### DETAILED DESCRIPTION

Stents are widely used in valve replacement and other medical procedures. To function properly, stents are required to be properly positioned and attached to the orifice after deployment from a delivery system, such as a balloon catheter that expands to deploy the stent or a retractable catheter that gradually retracts to permit the stent to assume an expanded form. As such, embodiments of the stent as set forth herein include particular properties and characteristics that address issues related to deploying and positioning the stent during valve replacement. First, the stent requires little expansion upon compression such that less force need be applied to the valve prosthesis attached to the stent. The stent also provides a stable, yet flexible scaffolding platform for the valve prosthesis because of the stent's ability to resist torsion, while also being capable of expanding and contracting over long periods of time. In addition, the geometry and mechanical properties of the stent allow for more anatomically-correct placement to properly fit into the orifice when the stent is initially positioned after deployment. Further details of the stent and other related components are discussed in greater detail below.

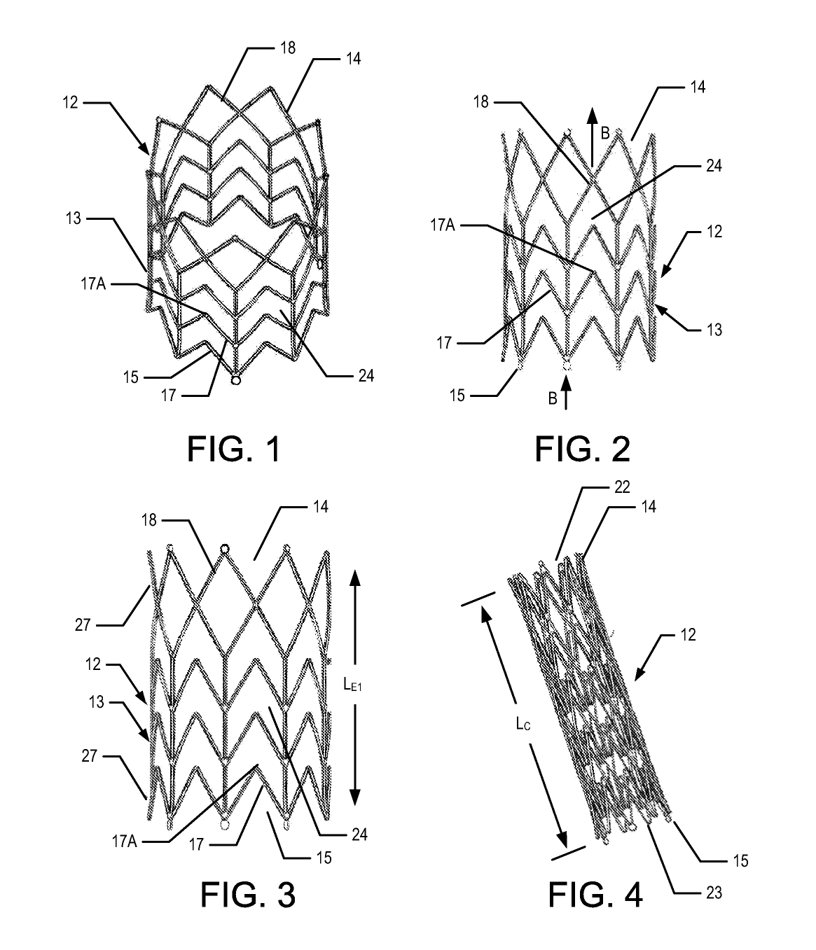

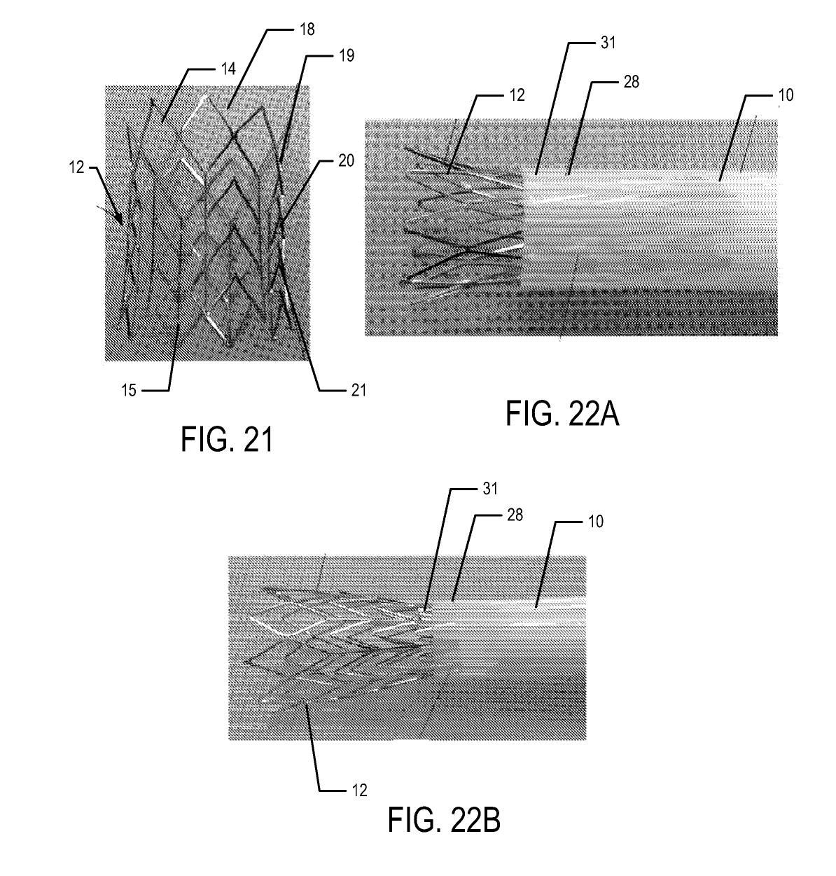

Referring to the drawings, an embodiment of an expandable stent attached to a valve prosthesis 11 for implantation and deployment by a delivery system 10 are illustrated and generally indicated as 12 in FIGS. 1-22. The valve prosthesis 11 is typically implanted in one of the channels or lumens of the body to replace a diseased natural valve. For example, the valve prosthesis 11 is attached to the stent 12 for implantation in the aorta during a valve replacement procedure. However, it is understood that it is possible to use the stent 12 without the valve prosthesis 11 in relation with implantation in other channels of the body by using the same technique as shall be discussed below for implantation of the cardiac valve prosthesis 11. For example, the procedure may include the implantation of: 1) a valve prosthesis 11 in the heart (for instance, a mitral valve, triscupid valve, aortic valve, or pulmonary valve) or vaculature; 2) a valve prosthesis 11 in the ureter and/or the vesica; 3) a valve prosthesis 11 in the biliary passages; and 4) a valve prosthesis 11 in the lymphatic system.

The following terms used in the detailed description will have the following meanings as set forth herein. As used herein, the term “chevron-shaped six-sided polygon” means a planar or non-planar figure that is bounded by a closed path or circuit, containing a sequence of six generally straight line segments, edges, or sides (i.e., by a closed polygonal chain) having six vertices or corners, wherein the interior of the polygon or body forms a generally chevron- shaped or “V” or inverted “V” shape. In the unfolded geometry of the stent 12, the chevron-shaped six-sided polygon is planar and the segments, edges, or sides are substantially straight. However, in its folded geometry, the stent 12 includes polygons that are not planar and may have segments, edges, or sides that are generally straight but can have substantial curvature to permit good approximation of the interior of the lumen or valve that the stent 12 is supporting and replacing.

As used herein, the term “self-expandable” means a material that is able to deform when a load is applied and return to its original shape when the load is removed without the use of an outside force. In the context of the stent 12, the stent 12 assumes a collapsed form to fit within the delivery system 10, but the stent 12 is able to return to its original fully expanded form only after the stent 12 is released from the delivery system 10.

As used herein, the term “expandable” shall mean a material that is able to deform when a load is applied, but will not return to its original shape when the load is removed. In the context of the stent 12, the stent 12 assumes a collapsed form to fit within the delivery system 10, but the stent 12 requires an exterior force, such as an expandable balloon, to exert a force to expand the stent 12.

As used herein, the term “passive” when used in reference to a marker, refers to the visibility of the marker based on the susceptibility artifacts, for example, dark spots on a magnetic resonance image (“MRI”), radiopaque markers in a fluoroscopy, dense spots in an X-ray, or echo in ultrasound generated by intrinsic properties (magnetic properties in the case of MRI, fluorescence in the case of fluoroscopy, absorption of X-ray photons in radiography, and sound in the case of ultrasound), of the marker.

As used herein, the term “active,” when used in reference to a marker, refers to the incorporation of an MRI receiver coil (for example, an antenna or guide wire, electrically connected to a scanner) into the delivery system 10, which is sensitive to signal only from adjacent tissue and is used to create bright spots on the MRI.

Referring to FIGS. 1-3, one embodiment of the stent 12 is shown in a fully expanded form prior to deployment from the delivery system 10; however, after deployment the stent 12 will expand to its environment to an expanded form that may be less than the fully expanded form. The stent 12 defines a proximal end 14 and a distal end 15 including a tubular lattice structure 13 having a radial direction and a longitudinal direction. In one embodiment, the tubular lattice structure 13 may be made from a material that has an elastic property that permits the stent 12 to self-expand from a collapsed form or bend when a force is applied to the stent 12, while in another embodiment, the stent 12 is made from a material that has an elastic, non-self expandable property that requires an exterior force be applied to expand the stent 12. An expandable balloon catheter (not shown) that exerts the necessary force required to expand the stent 12 may be utilized when the stent 12 is not self-expandable.

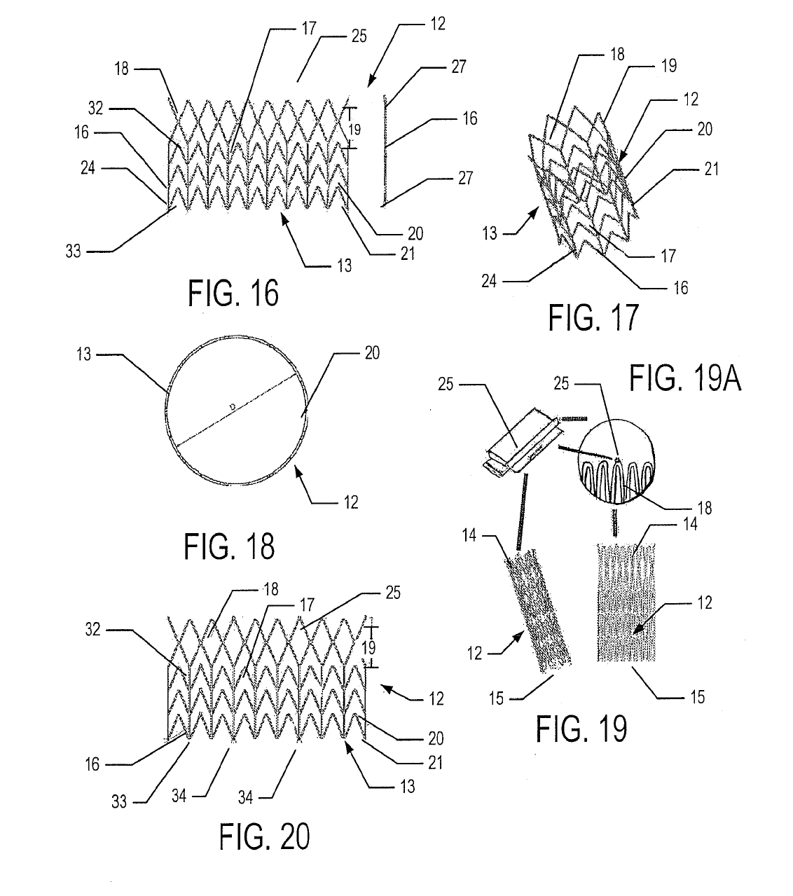

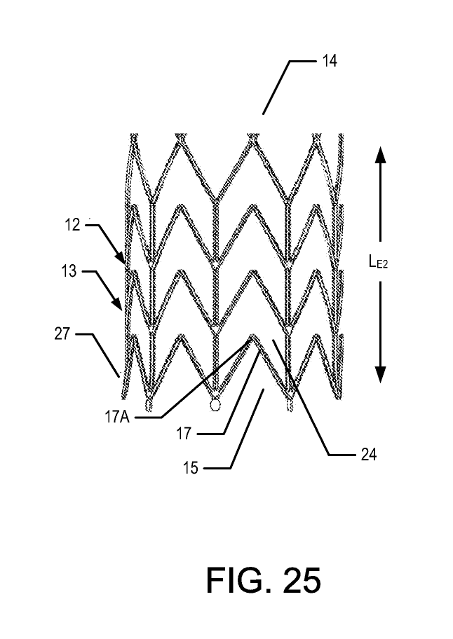

Referring to FIGS. 16 and 17, in one embodiment the tubular lattice structure 13 may define a proximal end zone 19, a middle zone 20 and a distal end zone 21. In another embodiment of the stent, designated 12A, that is shown in FIG. 25 the tubular lattice structure 13 may include only the middle zone 20 and distal zone 21 having only the six-sided polygons 24. The proximal end zone 19 may include a plurality of diamond-shaped four-sided polygons 18 that are interconnected together to form a crown shaped end portion along the proximal end 14 of the stent 12, while the middle and distal end zones 20 and 21 of the tubular lattice structure 13 include a plurality of interconnected chevron-shaped six-sided polygons 24. Each of the plurality of six-sided polygons 24 is collectively defined by a respective pair of rods 16 connected together by a respective pair of struts 17 that collectively form a chevron shape. The plurality of rods 16 are positioned substantially in the longitudinal direction B (FIG. 2) of the tubular lattice structure 13 and are in parallel orientation with respect to each other. As noted above, the tubular lattice structure 13 may include only the middle zone 20 and distal end zone 21 having only the chevron-shaped six-sided polygons 24 and not the four-sided polygons 18.

As shown in FIG. 3, the struts 17 of each six sided polygon 24 define a V-shape with an apex 17A formed by the strut 17 between each respective pair of rods 16 that may point toward the distal end zone 19 of the stent 12. In this configuration, the struts 17 may impart a barbed feel when a user runs their hand over the tubular lattice structure 16 from the proximal end 14 to the distal end 15 of the stent 12, while providing a relatively smooth feel when the user runs their hand from the distal end 15 to the proximal end 14 of the stent 12. Alternatively, the apex 17A may be oriented in the opposite direction towards the distal end zone 21. Moreover, the apex 17A of each strut 17 may point toward the direction of flow of fluid along longitudinal direction B (FIG. 2) through the stent 12 that also assists in the stabilization of the stent 12 after deployment due to the orientation of the struts 17. The struts 17 also provide structural reinforcement to the tubular lattice structure 13 that minimize the occurrence of fractures over time. In one embodiment, the proximal end zone 19 of the tubular lattice structure 13 may include 9 four-sided polygons 18, which allow the stent 12 to expand and conform to the shape of the orifice (not shown), for example, the aorta, over time. However, other embodiments of the stent 12 may have more or fewer than 9 four-sided polygons 18 that form the proximal end zone 19 of tubular lattice structure 13.

As shown in FIG. 4, the stent 12 may be placed in a collapsed form when constrained within the delivery system 10 prior to deployment and an expanded form (FIG. 8) after deployment from the delivery system 10 when the stent 12 expands to the limits imposed by its environment. FIG. 1, illustrates the stent 12 in a fully expanded form prior to being constrained within the delivery system 10. In one embodiment, the stent 12 in the collapsed form has substantially the same length (L_{C}) as the length (L_{E}) of the stent 12 in the fully expanded form due to the chevron-shaped six sided polygons 24 that constitute the tubular lattice structure 13. For example, the percentage that the stent 12 shortens the longitudinal length (L_{E2}) after deployment for the embodiment of the stent 12 with only the chevron shape six-sided polygons 24 is substantially 0%, while the percentage change in longitudinal length (L_{E}) that the embodiment of the stent 12 with both the four-sided polygons 18 and the six-sided polygons 24 is in a range between 0%-5%. In comparison, prior art stents have been found to have a change in longitudinal length of around 35% after expansion from the collapsed form. As such, the embodiments of the stent 12 do not substantially shorten when the stent 12 is deployed or lengthen when the stent 12 assumes a collapsed form when constrained within the delivery system 10. One advantage of maintaining substantially the same length of the stent 12 in either the collapsed or expanded form is that the valve prosthesis 11 is not stressed by the lengthening of the stent 12 during compression. In one embodiment, the stent 12 is made from at least one shape memory alloy, such as nickel-titanium alloy, including those alloys sold under the trade name of NITINOL. In the embodiment of the stent 12A that requires deployment by a balloon catheter, the stent 12A may be made from stainless steel, platinum/iridium, and magnesium.

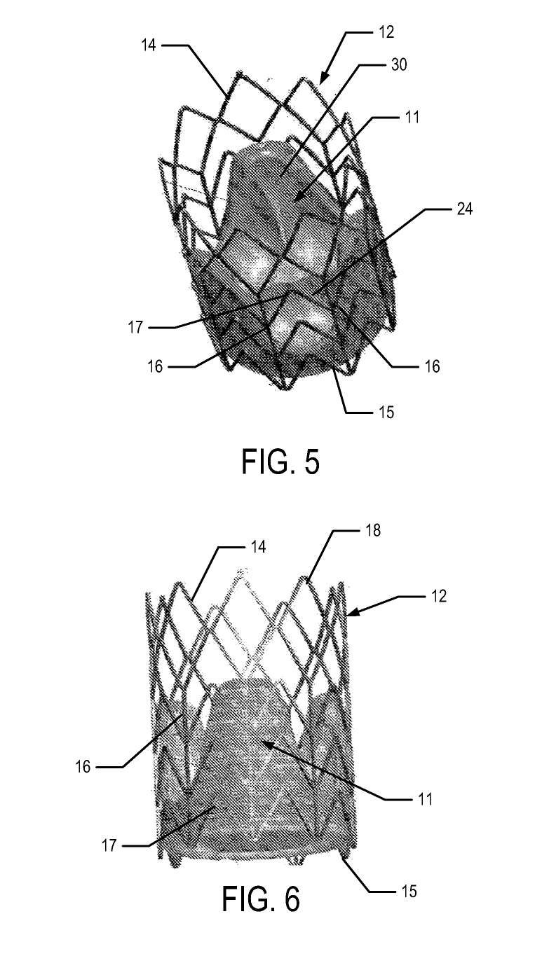

Referring to FIGS. 5 and 6, the stent 12 is shown in the fully expanded form with the valve prosthesis 11 disposed inside the stent 12. In one embodiment, the valve prosthesis 11 may be an aortic valve prosthesis having three commissures 30, wherein the commissures 30 are aligned with three of the rods 16 of the tubular lattice structure. The various embodiments of the valve prosthesis 11 may include a cardiac valve (including a mitral valve, tricuspid valve, aortic valve, or pulmonary valve), a vascular valve, a ureteral valve, a vesicle valve, a biliary passage valve, or a lymphatic system valve. In one embodiment, the stent 12 is crimped to the valve prosthesis 11, although in other embodiments the valve prosthesis 11 may be connected, affixed, crimped, or otherwise attached to the inner side of the tubular lattice structure 13 in any manner that securely engages the valve prosthesis 11 to the stent 12.

Referring to FIGS. 16, 18, 20 and 21, the stent 12 is shown in an unfolded geometric configuration. The values of the geometric parameters of the stent 12, for example, diameter (D), length (L), thickness (w), flare curvature (R), and rod width (ws) provide radial force and flexibility to the stent 12. For example, the values of the geometric parameters of the stent 12 when used for aortic valve replacement are as follows: the diameter (D) of the stent 12 needs to accommodate the typical diameters of the valve prosthesis 11 (For example, diameters of the valve prosthesis 11 of 21 mm, 23 mm, 25 mm, and 27 mm require a diameter (D) for the stent 12 to be 22 mm, 24 mm, 26 mm, and 28 mm, respectively); the length (L) has a range between 35 -37 mm); the thickness (w) is in a range of 0.35 mm-0.5 mm; the flare curvature (R) is in a range of 10-12 mm; the rod width (ws) is in a range of 0.35-0.5 mm. As shown in FIG. 3, each of the plurality of rods 16 define a first end 32 connected to one of the four-sided polygons 18 of the proximal end zone 19 and a second end 33 that forms a part of the distal end zone 21. Referring back to FIG. 16, the isolated view of the rod 16 shows that the first and second ends 33 and 34 defined by each rod 16 may define a respective flare 27 or slight bend in the rod 16. In addition, each rod 16 may be attached at only one point to the four-sided polygon 18 such that the rod 16 may not extend through the four-sided polygon 18 since the proximal end zone 19 of the tubular lattice structure 13 may become too rigid in such a configuration and may be less able to form the flare 27 required to properly seat the stent 12 after deployment, thereby preventing torsion of the stent 12 and valve prosthesis 11.

The flares 27 may be formed when the stent 12 assumes an expanded form after deployment from the delivery system 10. The degree that the flares 27 may bend is dependent on how much the stent 12 is allowed to expand after deployment in view of the environment, e.g., the diameter of the lumen may restrict the stent 12 from expanding to a fully expanded form. In addition, these flares 27 as well as the other geometric and mechanical parameters, such as the length (L) of the stent 12, discussed above allow for more anatomically-correct placement of the stent 12 as well as provide more flexible reinforcement/scaffolding of the prosthetic valve 11 by the stent 12. For example, the fracture of struts 17 may be minimized by virtue of the geometric and mechanical parameters of the tubular lattice structure 13.

When good visibility and monitoring is desired during deployment and positioning of the stent 12 the following methods may be utilized. Referring to FIGS. 16, 19, 19A and 20, one method involves affixing, such as by welding, a passive marker 25 to the distal end 14 of the stent 12. The passive marker 25 may include a high-density metal-containing material selected from the group consisting of gold, platinum, tantalum, stainless steel, and combinations thereof, which may be monitored, for example, by magnetic resonance imaging, X-ray imaging, fluoroscopy, or ultrasound. FIGS. 10 and 11 illustrate the image artifact in an MRI image that shows the location of the passive marker 25. In one embodiment, the stent 12 may have a protective insulating layer between the stent 12 and the passive marker 25 to prevent corrosion.

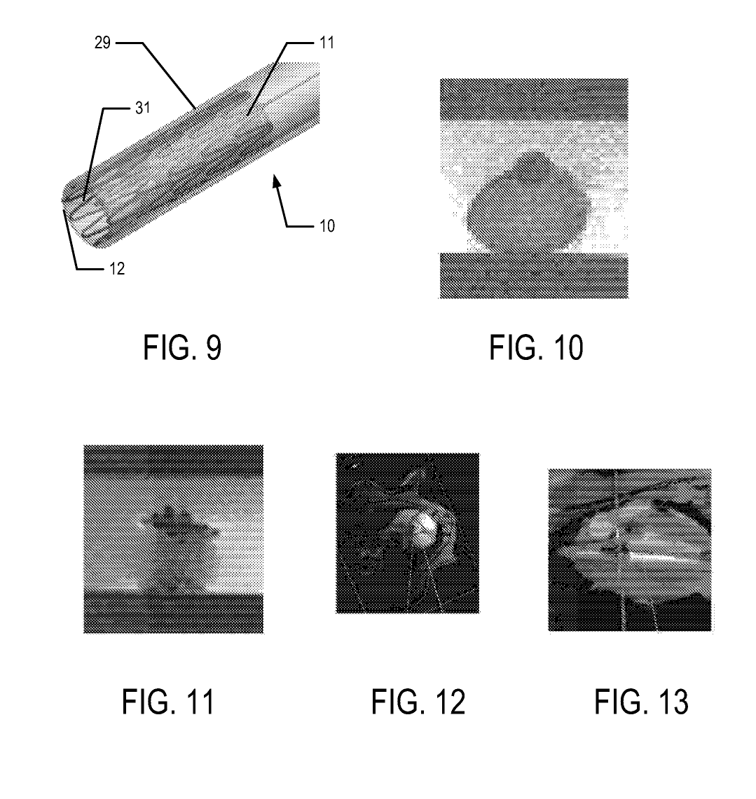

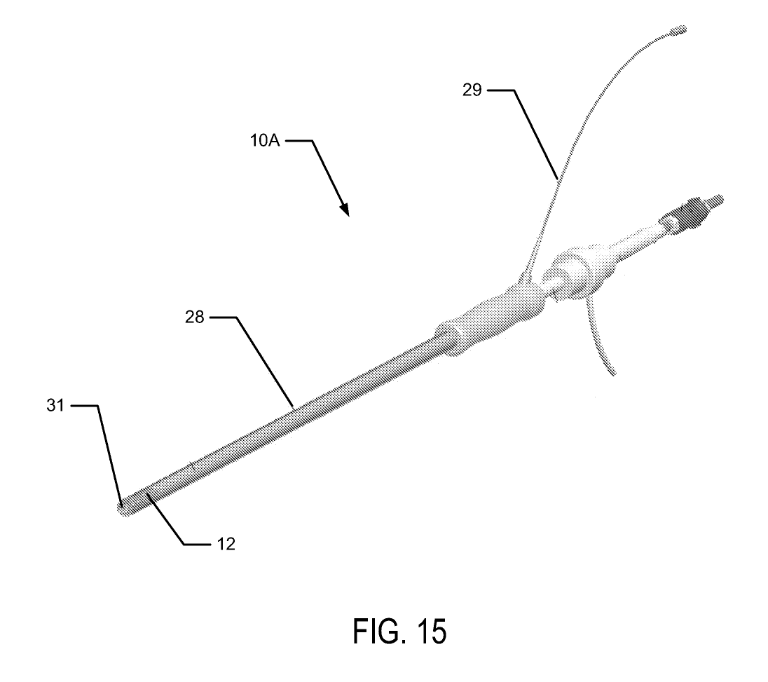

Another method may involve use of an active marker 29, such as an active guide wire shown in FIGS. 9 and 15, which is positioned along the inside wall of a retractable outer sheath 28, of the deployment system 10. FIGS. 12 and 13 show the colored trace of the active marker 29 shown on an MRI image. For example, the active guide wire 29 may be a loop coil antenna, manufactured using an insulated 0.005″ magnet copper wire. The coil length was adjusted to 1.1 inches with 0.026″ outer diameter. A 0.006″ profile twisted pair was used as a transmission lie for the loop coil antenna. The whole arrangement was insulated by using medical grade polyester heat shrink tubing and embedded inside the wall of a retractable outer sheath. The loop coil antenna was matched to 50 ohm and tuned to a Larmour frequency of a 1.5T MRI scanner.

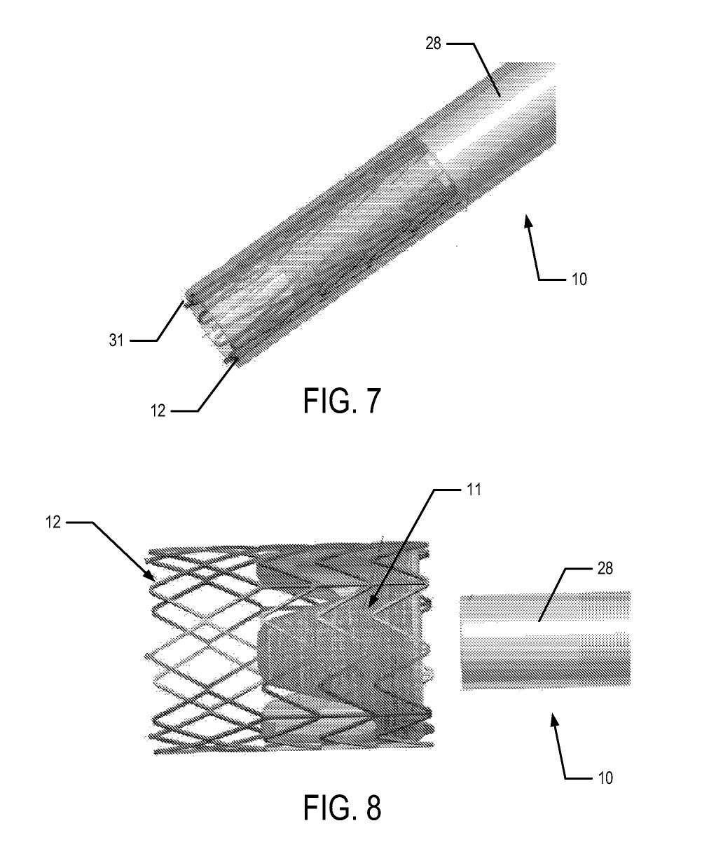

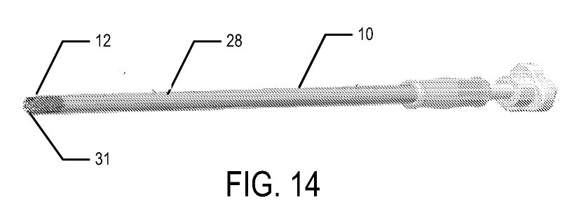

Referring to FIGS. 14 and 15, the stent 12 and valve prosthesis 11 may be adapted to be delivered, deployed, and positioned using at least two different embodiments of the delivery systems 10. For example, one embodiment, delivery system 10 (FIG. 14) may include a manually retractable outer sheath 28 that defines a sheath opening 31 with the stent 12 and valve prosthesis 11 disposed adjacent the opening 31. The delivery system 10 is manually actuated by the user to deploy the stent 12 and valve prosthesis 11 by operation of the handle such that the outer sheath 28 is gradually retracted which incrementally deploys the stent 12 from a collapsed form shown in FIG. 7 to partial deployment shown in FIGS. 22A and 22B until the stent 12 is fully deployed as illustrated in FIG. 8. In an alternate embodiment of the delivery system shown in FIG. 15, designated 10A, a robotic delivery system 10A operates in a similar manner as the other embodiment of the delivery system 10 except the robotic delivery system 10A may deploy the stent 12 using a mechanism that automatically retracts the outer sheath 28 rather than manually retracting the outer sheath 28. In yet another embodiment, the delivery system 10 may be a balloon catheter with an expandable balloon (not shown) that is disposed within the stent 12 such that expansion of the balloon by inflation causes the stent 12 to assume an expanded form during deployment.

Referring back to FIG. 20, one embodiment of the stent 12 may include one or more grasping members 34, such as spherical beads or the like, that provide retracting capability to the stent 12 by providing one or more structural elements capable of being grasped by a loop catheter or loop snare wire (not shown) for retaining the stent 12 and valve prosthesis 11 and retract the outer sheath 28. The loop snare wire system prevents early or accidental deployment of the valve prosthesis 11 as well as provides a means for repositioning the stent 12 if the stent 12 has not yet been completely deployed.

In one embodiment, the stent 12 and valve prosthesis 11 may be deployed by percutaneously delivering the stent 12 and valve prosthesis 11 disposed within the outer sheath 28 to a location in the lumen that requires either repair or replacement. The user then retracts the outer sheath 28 using the delivery system 10 such that the stent 12 is incrementally deployed from the collapsed form to the fully expanded form. Once the stent 12 is deployed, the user may then monitor the orientation and location of the stent 12 in the lumen using the passive marker 25. If desired, the user may use a maker, such as the passive marker 25 to reposition the orientation and location of the stent 12 by engaging and manipulating the grasping member 34 of the stent 12. However, once the stent 12 is completely deployed, the stent 12 cannot be repositioned or reoriented.

### EXAMPLE

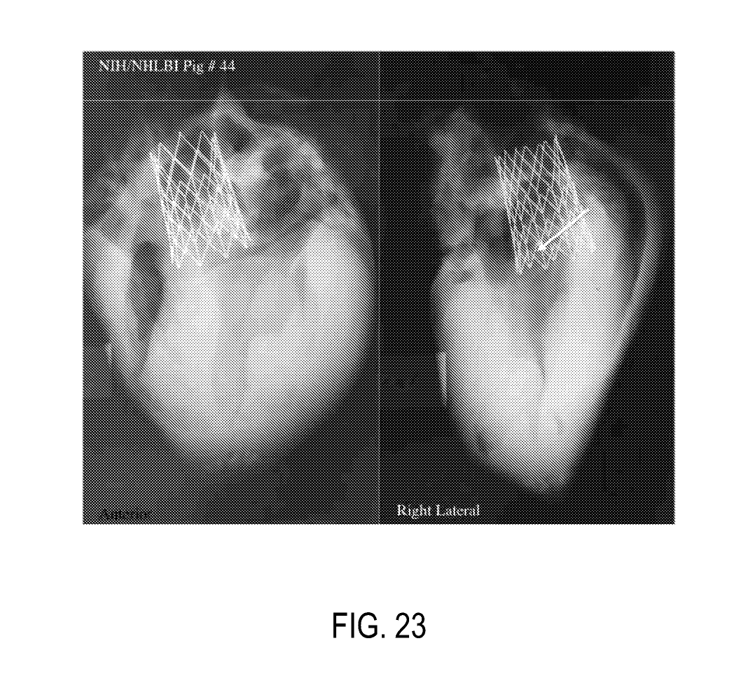

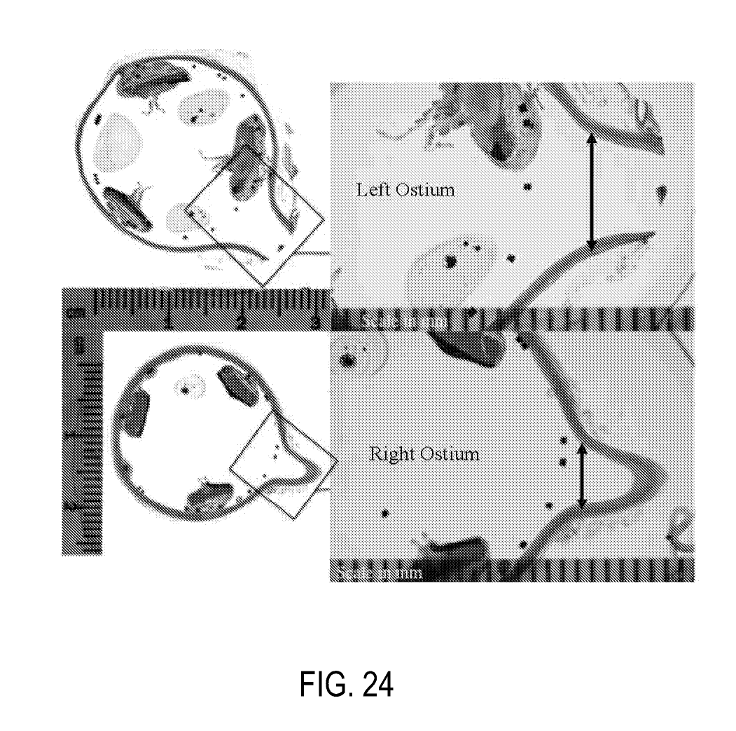

Tests were performed using the self-expandable embodiment of the stent 12 to test its structural integrity after deployment and positioning within the orifice. Specifically, one porcine heart with a prosthetic aortic valve was implanted into the aortic root for histopathologic evaluation. Referring to FIG. 23, the radiographs of the heart and aortic root show a widely and evenly expanded stent frame. The right lateral radiographic image also disclosed a single strut fracture on the proximal crown. Grossly, the stent appeared to be properly seated in the aortic root. The right cusp was centered on the right coronary ostium with the prosthetic annulus 0.5 cm inferior to the ostium. The left cusp was rotated posteriorly, aligning the left ostium evenly with the anterior base of the leaflet. Both coronary ostia were widely patent and unobstructed by the stent frame. The proximal end of the stent frame was covered and well seated over the native aortic valve annulus with no gaps between the stent frame, annulus or aortic root. The prosthetic annulus was covered with an opaque fibrous tissue overgrowth. One bare stent crown tip was noted on the anterior lateral wall. Distally, the stent was well apposed to the aortic wall with most struts covered with translucent neointimal overgrowth with the exception of the struts adjacent to the coronary ostia. FIG. 24 also illustrates that only one strut 17 fracture occurred after the stent was deployed, which was an unexpected result in view of prior art stents that would have multiple struts that fractured over time.

The average strut fractures for a platinum-iridium stent 12 after an implantation of 6 months was 5.0±3.1 (mean±std. dev.), while the average fractures for stent 12 was 1.6±2.5 (mean±std. dev.). The fractures were due to the material fatigue of the stent 12 and the expansion, contraction, torsion forces generated between the aorta and the stent 12. The platinum-iridium stent 12 had more strut fractures, while the NITINOL self-expanding stent 12A had fewer or no strut fractures (p=0.046).

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. A stent (12A) comprising:
a tubular lattice structure (13) having a radial direction and a longitudinal direction, the tubular lattice structure (13) comprising:
a middle zone (20) in communication with a distal end zone (21), the middle zone (20) and distal end zone (21) including a plurality of rods (16) positioned substantially in the longitudinal direction of the tubular lattice structure (13) and interconnected by a plurality of struts (17) that collectively define a plurality of six-sided polygons (18).

2. The stent (12A) of claim 1, wherein each of the plurality of struts (17) defines an apex (17A) that is oriented towards the proximal end zone (19).

3. The stent (12) of claim 1, wherein the tubular lattice structure (13) further comprises a proximal end zone (19) in communication with the middle end zone (13), wherein the proximal end zone (19) includes a plurality of interconnected four-sided polygons (18).

4. The stent of (12A) claim 1, wherein the tubular lattice structure (13) is collapsible and expandable.

5. The stent (12A) of claim 4, wherein the tubular lattice structure (13) assumes a collapsed form from a fully expanded form when being placed in a constrained position and from the collapsed form to an expanded form after deployment.

6. The stent (12A) of claim 5, wherein the tubular lattice structure (13) has substantially the same longitudinal length in the collapsed form and in the expanded form.

7. The stent (12A) of claim 1, wherein the tubular lattice structure (13) is formed from at least one shape memory alloy.

8. The stent (12A) of claim 1, wherein the stent further comprises at least one marker affixed to the tubular lattice structure (13).

9. The stent (12A) of claim 1, wherein the tubular lattice structure (13) further comprises a grasping member (34) connected to at least one of the plurality of rods (16).

10. The stent (12A) of claim 1, wherein each of the plurality of rods (16) defines a first end (32) and a second end with each of the first and second ends defining a flare.

11. The stent (12A) of claim 10, wherein the first end (33) of each of the plurality of rods (16) is connected to one of the four-sided polygons (18) of the proximal end zone (19) and the second end (33) of each of the plurality of rods (16) forms a part of the distal end zone (21).

12. The stent (12A) of claim 1, further comprising a prosthesis (11) disposed inside the tubular lattice structure (13) of the stent (12).

13. The stent (12) of claim 1, wherein the stent (12) further comprises a protective insulated coating.

14. The stent (12A) of claim 12, where the prosthesis (11) includes a plurality of commissures (30), and wherein the prosthesis (11) is disposed and oriented inside the tubular lattice structure (13) such that each respective plurality of commissures (30) are aligned with at least one of the plurality of rods (16).

15. The stent (12A) of claim 1, wherein the tubular lattice structure (13) is formed from a group consisting of at least stainless steel, platinum/iridium, and magnesium.

16. A delivery system (10) comprising:
a hollow outer sheath (28) defining an opening (31);
a stent (12A) disposed adjacent the opening (31) of the hollow catheter sheath (28) and in a collapsed form, the stent (12A) including a tubular lattice structure (13) having a radial direction and a longitudinal direction, the tubular lattice structure (13) comprising:
a middle zone (20) in communication with a distal end zone (21), the middle zone (20) and distal end zone (21) including a plurality of rods (16) positioned substantially in the longitudinal direction of the tubular lattice structure (13) and interconnected by a plurality of struts (17) that collectively define a plurality of six-sided polygons; and
a valve prosthesis (11) disposed inside the tubular lattice structure (13) of the stent (12A).

17. The delivery system (10) of claim 16, wherein the tubular lattice structure (13) further comprises:
a proximal end zone (19) in communication with the middle end zone (13), wherein the proximal end zone (19) includes a plurality of interconnected four-sided polygons (18).

18. The delivery system (10) of claim 16, further comprising a marker (25, 29) affixed to the tubular lattice structure (13) for providing a visual indicator as to the location of the stent (12A).

19. The delivery system (10) of claim 18, wherein the marker (25, 29) is a passive marker (25).

20. The delivery system (10) of claim 18, wherein the marker (25, 29) is an active marker (29).

21. The delivery system (10) of claim 17, wherein the hollow outer sheath (28) is retractable for deploying the stent (12A).

22. A method for delivering and repositioning a stent (12A) in a lumen comprising:
providing a stent (12A) including a tubular lattice structure (13) having a radial direction and a longitudinal direction, the tubular lattice structure (13) comprising:
a middle zone (20) in communication with a distal end zone (21), the middle zone (20) and distal end zone (21) including a plurality of rods (16) positioned substantially in the longitudinal direction of the tubular lattice structure (13) and interconnected by a plurality of struts (17) that collectively define a plurality of six-sided polygon (18) in a delivery system (10), the delivery system (10) having a hollow, retractable hollow outer sheath (28) defining an opening (31) with the stent (12A) disposed therein adjacent the opening (31);
constraining the stent (12A) in a collapsed form within a delivery system (10) including a hollow outer sheath (28) adapted to receive the stent (12A) therein in the collapsed form;
delivering the stent (12A) percutaneously to a location in a lumen that requires repair or replacement;
retracting the outer sheath (28) of the deployment system (10) relative to the stent (12A) and permitting the stent (12A) to expand from the collapsed form to an expanded form in the location; and
monitoring an orientation and the location of the stent (12A) in the lumen.

23. The method of claim 22, wherein the stent (12A) includes a marker (25, 29) and further comprising:
repositioning the stent (12) using the marker (25, 29) to visually indicate the position of the stent (12) within the lumen.

24. The method of claim 22, wherein the tubular lattice structure (13) further comprises:
a proximal end zone (19) in communication with the middle end zone (20), wherein the proximal end zone (19) includes a plurality of interconnected four-sided polygons (18).

25. The method of claim 22, wherein one or more grasping members (34) are affixed to the tubular lattice structure (13) for manipulation by the delivery system (10).
